(19) United States
(12) Patent Application Publication
(10) Pub. No.: US 2011/0027324 A1
(43) Pub. Date: Feb. 3, 2011

(54) O-DESMETHYL-VENLAFAXINE FOR TREATING MAJOR DEPRESSIVE DISORDER

(75) Inventors: Robert Michael Poole, Devon, PA (US); Karen Astrid Tourian, Philadelphia, PA (US)

Correspondence Address: CHOATE, HALL & STEWART LLP/WYETH LLC, PATENT GROUP, TWO INTERNATIONAL PLACE, BOSTON, MA, 02110, US

(73) Assignee: Wyeth LLC, Madison, NJ (US)

(21) Appl. No.: 12/747,096

(22) PCT Filed: Dec. 10, 2007
(86) PCT No.: PCT/US2007/086986

§ 371 Date: Oct. 5, 2010

(51) Int. Cl. A61K 9/00; A61K 31/137; A61P 25/24

(52) U.S. Cl. 424/400; 514/653

(57) ABSTRACT

The present invention provides methods for treating MDD. In general, the methods comprise administering to a patient in need thereof a daily dose of about 50 mg ODV or an equivalent amount of a pharmaceutically acceptable salt thereof. In certain embodiments, a patient in need of treatment is characterized by a primary diagnosis of MDD. In some embodiments, the dose is administered as a single daily dose. In one set of embodiments, the methods involve administering an oral dosage form comprising the succinate salt of ODV.

### BACKGROUND OF THE INVENTION

Major depressive disorder (“MDD”) is a largely untreated psychiatric disorder that presents a serious clinical problem and reduces productivity and quality of life while increasing mortality. MDD is one of the most common problems encountered in primary care, affecting 6% to 10% of all patients who present in this setting. In the community-dwelling elderly population between 1-3% of patients have MDD, with depressive symptoms being present in approximately 15% of patients. MDD is also a common and disabling complication of the postpartum period in women.

The depression of MDD worsens the prognosis for other coexisting medical problems and may even lead to patient suicide. Fifteen percent of patients with severe MDD die by suicide. The societal costs of MDD that include costs of treatment, morbidity, and lost productivity of patients afflicted with MDD have been estimated at >$43 billion annually in the United States alone. MDD is an increasingly major source of disability worldwide which is predicted to become second only to ischemic heart disease by the year 2020. There is a need in the art for methods of treating MDD.

### SUMMARY OF THE INVENTION

The present invention provides methods for treating MDD that encompass our finding that a daily dose of about 50 mg O-desmethyl-venlafaxine (“ODV”) or an equivalent amount of a pharmaceutically acceptable salt thereof can be used to treat MDD. In general, the methods comprise administering to a patient in need thereof a daily dose of about 50 mg ODV or an equivalent amount of a pharmaceutically acceptable salt thereof. In certain embodiments, a patient in need of treatment is characterized by a primary diagnosis of MDD. In some embodiments, the dose is administered as a single daily dose. In one set of embodiments, the methods involve administering an oral dosage form comprising the succinate salt of ODV.

### DETAILED DESCRIPTION OF CERTAIN EMBODIMENTS OF THE INVENTION

O-desmethyl-venlafaxine is a major metabolite of venlafaxine and has been shown to inhibit norepinephrine and serotonin uptake. Klamerus et al., “Introduction of the Composite Parameter to the Pharmacokinetics of Venlafaxine and its Active O-Desmethyl Metabolite”, J. Clin. Pharmacol. 32:716-724 (1992). O-desmethyl-venlafaxine, chemically named 1-[2-(dimethylamino)-1-(4-phenol)ethyl]-cyclohexanol, was exemplified as a fumarate salt in U.S. Pat. No. 4,535,186 and as a free base in PCT Patent Publication No. WO 00/32555. O-desmethyl-venlafaxine succinate (“ODV succinate”) was first described in U.S. Pat. No. 6,673,838 continued in U.S. Pat. No. 7,026,508 both incorporated herein by reference.

### Methods of Treatment

The present invention encompasses results from clinical studies of the efficacy and safety of daily doses of 50 and 100 mg ODV in the treatment of MDD (see Examples). The results of these studies show that 50 mg ODV has a safety profile similar to that observed in short-term (8 weeks) and long-term (from 6 months up to 12 months) studies in which doses ranged from 100 to 400 mg/day. The results of two of three studies also reveal that a daily dose of 50 mg can be as effective as a 100 mg dose in treating MDD.

The present invention takes advantage of these results by providing an improved method for treating MDD which comprises administering to a patient in need thereof a daily dose of about 50 mg of ODV or an equivalent amount of a pharmaceutically acceptable salt thereof. It is to be understood that the term “treating” is used herein to refer to situations in which the desired symptoms or disorder are ameliorated. This encompasses both prophylactic and therapeutic situations. The term “about” is used herein to mean within 15%, preferably within 10%, and more preferably within 5% of a given value or range. In addition, the term “equivalent amount” refers to a weight quantity of a pharmaceutically acceptable salt of ODV that is an equal molar quantity, as understood by one of ordinary skill in the art, to about 50 mg of ODV free base.

It will be appreciated that the methods may be used to treat any patient suffering or likely to suffer from depression. In certain embodiments, a patient in need of treatment is or has been characterized by a primary diagnosis of MDD (e.g., based on the criteria in the Diagnostic and Statistical Manual of Mental Disorders, 4^{th} edition (DSM-IV)). For example, MDD may be diagnosed on the basis of five of the following nine symptoms (one being a depressed mood or loss of interests/pleasure), present most of the day nearly every day for a minimum of two consecutive weeks: depressed mood, loss of interests/pleasure, change in sleep, change in appetite or weight, change in psychomotor activity, loss of energy, trouble concentrating, thoughts of worthlessness or guilt, and thoughts about death or suicide. In some embodiments, a patient in need of treatment is characterized by at least five of these depressive symptoms for at least 30 days. In one embodiment, a patient in need of treatment may have only experienced a single depressive episode. In another embodiment, a patient in need of treatment may have experienced recurrent depressive episodes. In one embodiment, a patient demonstrates minimum screening and baseline scores of 20 on the Hamilton Rating Scale for Depression, 17-item (HAM-D₁₇), of 2 on item 1 (depressed mood) on the HAM-D₁₇, and of 4 on the Clinical Global Impressions Scale-Severity (CGI-S).

In certain embodiments, the oral dose is administered as a single dose of about 50 mg. It will be appreciated that ODV may also be administered as multiple doses (e.g., two doses of about 25 mg, five doses of about 10 mg, etc.). When administered as multiple doses it is to be understood that each dose can be administered at any time within the daily period. For example, in one embodiment two doses of about 25 mg may be administered simultaneously. In another embodiment, two doses of about 25 mg may be administered sequentially, e.g., separated by a 1, 2, 4, 6, 8 or 12 hour interval.

In certain embodiments, a higher daily dose, e.g., 100, 200 or 400 mg may be administered for a period of time during the treatment period. These higher doses may be appropriate if a patient does not respond to a daily dose of 50 mg.

In one embodiment, the daily dose is administered continuously for a treatment period of 2 weeks, 1 month, 2 months, 3 months, 4 months or more. In certain embodiments, the dose is gradually increased during a titration period. For example, a daily dose of 10, 25, 50 and optionally 100 mg may be administered over successive days in order to initiate treatment. In certain embodiments, the dose is gradually decreased during a tapering period upon discontinuation of treatment. For example, a daily dose of 25, 10 and optionally 0 g may be administered over successive days in order to discontinue treatment. It will be appreciated that the titration and tapering periods may include different phases, e.g., a first phase during which a daily dose of 10 mg, a second phase during which a daily dose of 25 mg is administered, etc. (or vice-versa when considering a tapering period). Each phase may last the same or a different length of time (e.g., 1-4 days). Others will readily envisage variations on these exemplary dosage schemes. In one embodiment a placebo (i.e., 0 mg ODV) can be administered during part or all of a titration or tapering period. In certain embodiments, a titration or tapering period may be as short as 1 day and as long as 1 month, e.g., it may last 1, 2, 3, 4, 5, 6, 7, 8, 9, 10 etc. days. For example, a titration or tapering period may last 4 to 10 days, e.g., 7 days.

In one embodiment, patients with severe renal impairment (24 hr CrCl<30 ml/min) and/or end-stage renal disease are administered a daily dose of 50 mg every other day throughout their treatment period.

If a patient has recently discontinued treatment with a monoamine oxidase inhibitor (MAOI), a period of 14 days should elapse prior to initiation of therapy with ODV. Similarly, a period of 7 days should elapse after stopping ODV before starting an MAOI.

### Oral Dosage Forms

In certain embodiments, the methods may comprise administering an oral dosage form comprising O-desmethyl-venlafaxine or a pharmaceutically acceptable salt thereof and one or more pharmaceutically acceptable excipients. In some embodiments, the methods may comprise administering an oral dosage form which comprises ODV succinate. ODV succinate may be formed by any method known in the art, e.g., by contacting stoichiometric amounts of succinic acid with ODV free base as described in U.S. Pat. Nos. 6,673,838 and 7,026,508. ODV free base may be prepared by any method known in the art, e.g., according to the general procedures outlined in U.S. Pat. No. 4,535,186 or by demythelation of venlafaxine as described in U.S. Pat. No. 7,026,508. Venlafaxine may be prepared in accordance with procedures known in the art, such as those described in U.S. Pat. No. 4,535,186, which is incorporated herein by reference.

The oral dosage form also includes one or more pharmaceutically acceptable excipients, e.g., one or more fillers, lubricants, glidants, rate controlling polymers, or combinations thereof. One skilled in the art will readily appreciate that the category under which a particular excipient is listed is not intended to be limiting; in some cases a particular excipient might appropriately fit it more than one category. In addition, as will be appreciated, the same excipient can sometimes perform different functions, or can perform more than one function, in the context of a particular dosage form, for example depending upon the amount of the excipient and/or the presence of other excipients and/or active compound(s).

In certain embodiments, an oral dosage form comprises a tablet which comprises ODV, or an equivalent amount of a pharmaceutically acceptable salt thereof, a filler, a lubricant, a glidant, and a rate controlling polymer. According to some embodiments, an oral dosage form may also include a non-functional coating (e.g., a tablet coating). A “non-functional coating” is a coating that does not significantly affect the release characteristic(s) of ODV from the oral dosage form when administered. Examples of a non-functional coat include a seal coat (e.g., hydroxypropyl cellulose, hypromellose or polyvinyl alcohol). In certain embodiments, a non-functional coating is a polish coat or seal coat. In one embodiment, the tablet is taken whole without crushing of breaking. In one embodiment, the tablet is taken with food. In another embodiment, the tablet is taken without food.

### Rate Controlling Polymer

In certain embodiments, an oral dosage form of the provided method of treatment is a sustained release oral dosage form, e.g., a tablet. The sustained release oral dosage form may comprise ODV or a pharmaceutically acceptable salt thereof and one or more rate controlling polymers (i.e., a polymeric material which controls the rate at which the ODV is released once administered). Typically, the sustained release oral dosage form provides therapeutically effective plasma levels of ODV over at least a 16 to 20 hour period.

Any polymer that controls the release of active ingredients such as ODV once administered may be used, e.g., see “Remington: The Science and Practice of Pharmacy”, Lippincott Williams & Wilkins, 21″ Edition (2005), which is incorporated herein by reference. As is well known in the art, suitable rate controlling polymers include, but are not limited to, a hydroxyalkyl cellulose, such as hydroxypropyl cellulose or hydroxypropyl methyl cellulose (HPMC); poly(ethylene)oxide; an alkyl cellulose, such as ethyl cellulose or methyl cellulose; carboxymethyl cellulose; hydrophilic cellulose derivatives; polyethylene glycol, etc. In some embodiments, the oral dosage form comprises from about 10% to about 30% by weight of ODV or an equivalent amount of a pharmaceutically acceptable salt thereof and from about 50% to about 70% by weight of one or more rate controlling polymers. In one embodiment, the rate controlling polymer is hydroxypropyl methyl cellulose (HPMC).

### Filler

In certain embodiments, the oral dosage form may include an amount of filler. Suitable fillers (also referred to in the art as “diluents” and/or “binders”) arc well known in the art, e.g., see “Remington: The Science and Practice of Pharmacy”, Lippincott Williams & Wilkins, 21′ Edition (2005). For example, common fillers include but are not limited to starch, PVP (polyvinyl pyrrolidone), low molecular weight HPC (hydroxypropyl cellulose), microcrystalline cellulose (e.g., Avicel®), silicified microcrystalline cellulose (Prosolv 50), low molecular weight HPMC (hydroxypropyl methylcellulose), low molecular weight carboxymethyl cellulose, ethylcellulose, alginates, gelatin, polyethylene oxide, acacia, dextrin, sucrose, magnesium aluminum silicate, and polymethacrylates. Fillers include agents selected from the group consisting of microcrystalline cellulose (e.g., Avicel®), starch, lactitol, lactose, a suitable inorganic calcium salt, sucrose, glucose, mannitol, silicic acid, or a combination thereof. In some embodiments, the oral dosage form comprises from about 10% to about 30% by weight of ODV or an equivalent amount of a pharmaceutically acceptable salt thereof and about 5% to about 15% filler, based upon total weight of given oral dosage form. In certain embodiments, the filler is microcrystalline cellulose.

### Lubricant

In certain embodiments, an oral dosage form of given method of treatment may comprise a lubricant. Lubricants, generally, are substances used in solid dosage forms to reduce friction during compression. Such compounds include, by way of example and without limitation, sodium oleate, sodium stearate, calcium stearate, zinc stearate, magnesium stearate, polyethylene glycol, talc, mineral oil, stearic acid, sodium benzoate, sodium acetate, sodium chloride, and other materials known to one of ordinary skill in the art, e.g., see “Remington: The Science and Practice of Pharmacy”, Lippincott Williams & Wilkins, 21^{st} Edition (2005). In some embodiments, the oral dosage form comprises from about 10% to about 30% by weight of ODV or an equivalent amount of a pharmaceutically acceptable salt thereof and about 0.5% to about 2% lubricant, based upon total weight of given oral dosage form. In certain embodiments, the lubricant is magnesium stearate.

### Glidant

In some embodiments, an oral dosage form of provided method of treatment may comprise a glidant. Glidants are substances that are generally used to improve the flow characteristics of granulates and powders by reducing interparticulate friction. In some embodiments, the glidant component, comprises one or more of talc, silicon dioxide (e.g., colloidal silicon dioxide), silica gel, asbestos free talc, sodium aluminosilicate, calcium silicate, powdered cellulose, microcrystalline cellulose, sodium benzoate, calcium carbonate, magnesium carbonate, metallic stearates, calcium stearate, magnesium stearate, zinc stearate, stearowet C, magnesium lauryl sulfate, magnesium oxide, and mixtures thereof. In some embodiments, the oral dosage form comprises from about 10% to about 30% by weight of ODV or an equivalent amount of a pharmaceutically acceptable salt thereof and about 2% to about 4% glidant, based upon total weight of given tablet. In certain embodiments, the glidant is talc.

### Coating

In certain embodiments, an oral dosage form of given method of treatment may comprise a non-functional coating. For example, in some embodiments, a tablet may comprise a non-functional coating. In some embodiments, the non-functional coating is a seal coat. For example, a suitable seal coating can be applied as a solution (e.g., Opaglos® 2 solution). Upon drying, seal coating may be from about 10% to about 30% by weight of ODV or an equivalent amount of a pharmaceutically acceptable salt thereof and about 3% to about 5% of weight gain of the total coated dosage form. In certain embodiments, the non-functional coating is Opaglos® 2.

### Production

An oral dosage form of given method of treatment may be prepared by any known method, e.g., see “Remington: The Science and Practice of Pharmacy”, Lippincott Williams & Wilkins, 21′ Edition (2005). In some embodiments, these methods may include a roller compaction step. For example, a dry granulate which comprises ODV, or a pharmaceutically acceptable salt thereof, and one or more excipients may be treated by roller compaction and then compressed into tablets. In some embodiments, a non-functional coating may also be applied to a compressed tablet as is known in the art.

### Unit Dosage Form

In some embodiments, the oral dosage form is in unit dosage form, e.g., in the form of one or more tablets, capsules, caplets, etc. In such form, the oral dosage form is sub-divided into unit doses containing appropriate quantities of ODV, or a pharmaceutically acceptable salt thereof. The unit dosage form can be, for example, a tablet itself, or it can be the appropriate number of any such dosage forms in package form.

In some embodiments, an oral dosage form of given method of treatment is a tablet comprising about 50 mg ODV, or an equivalent amount of a pharmaceutically acceptable salt thereof. In certain embodiments, such tablets comprise 50 mg of ODV, or an equivalent amount of a pharmaceutically acceptable salt thereof.

In some embodiments, an oral dosage form of given method of treatment includes two tablets each comprising about 25 mg ODV, or an equivalent amount of a pharmaceutically acceptable salt thereof. In certain embodiments, such tablets each comprise 25 mg of ODV, or an equivalent amount of a pharmaceutically acceptable salt thereof.

### EXAMPLES

### Example 1

### Tablets

The ingredients used in preparing 50 mg ODV succinate tablets that were used in subsequent examples are provided in Table 1.

TABLE 1

| Ingredient | % w/w | Input/Tablet (mg) |
|---|---|---|
| Intra Granular | | |
| ODV succinate | 21.27 | 75.87¹ |
| Microcrystalline Cellulose | 8.05 | 28.71 |
| HPMC | 56.25 | 200.66 |
| Talc | 1.63 | 5.83 |
| Magnesium Stearate | 0.30 | 1.07 |
| Extra Granular | | |
| HPMC | 6.25 | 22.30 |
| Talc | 1.50 | 5.35 |
| Magnesium Stearate | 0.90 | 3.21 |
| Film Coat | | |
| Opaglos ® 2(pink) | 3.85 | 13.73 |
| Tablet | 100 | 356.73 |

¹Equivalent to 50 mg ODV.

Tablets were prepared from these ingredients as follows:
1. The following materials were first passed through an appropriate screen and transferred into a diffusion blender (e.g. bin blender): ODV succinate, HPMC (a portion), microcrystalline cellulose, and talc (portion). The materials were blended.
2. A portion of the magnesium stearate was then added through an appropriate screen to the blender. The materials were blended.
3. The dry granulation blend was then discharged into the hopper of a roller compactor. The blend was compacted using a smooth (top) and a knurled (bottom) roller combination. The blend was then passed through an appropriate screen milling system before the material was collected in a diffusion blender.
4. The remaining HPMC and talc were added into the diffusion blender and mixed.
5. The remaining magnesium stearate was added into this blender and mixed.
6. The material was then compressed using an appropriate rotary tablet press and tablets were collected in suitable containers.

The film coating was prepared and applied to these tablets as follows:
7. Purified water was added into an appropriate mixer tank and agitated with a variable speed mixer.
8. Opaglos® 2 powder was added to the water while mixing. The suspension was maintained at ambient temperature with continuous mixing during the film coating process.
9. The tablet cores were placed into the perforated coating pan.
10. Sufficient amounts of the Opaglos® 2 film coat suspension were then added to obtain the desired dry coat weight gain.
11. The coated tablets were unloaded into appropriate containers.

10, 25 and 100 mg tablets are prepared using a similar process and adjusted amounts of each ingredient in Table 1. It will be appreciated that in certain embodiments, each ingredient may be present in a dose proportional amount. The present invention encompasses tablets that are prepared with variations on dose proportional amounts of one or more excipients.

### Example 2

### MDD Study No. 1

### Study Design

This example describes the results of a multicenter, randomized, double-blind, placebo-controlled, parallel-group study that was designed to evaluate the efficacy and safety of 50 and 100 mg/day ODV for treating MDD.

Subjects were male or female outpatients at least 18 years of age who had depressive symptoms for at least 30 days before the screening visit, and a primary diagnosis of MDD based on the criteria in the Diagnostic and Statistical Manual of Mental Disorders, Fourth edition (DSM-IV), single or recurrent episode, without psychotic features. Minimum screening and baseline scores of 20 on the Hamilton Rating Scale for Depression, 17-item (HAM-D₁₇), of 2 on item 1 (depressed mood) on the HAM-D₁₇, and of 4 on the Clinical Global Impressions Scale-Severity (CGI-S) were also required.

The planned enrollment was 480 subjects. A total of 703 subjects were screened for participation; 229 were screen failures and 474 were randomly assigned to treatment: 159 were assigned to receive placebo, 158 were assigned to receive ODV 50 mg/day; and 157 were assigned to receive ODV 100 mg/day. Twenty-three (23) of these 474 subjects were considered “no-data subjects” (randomized to treatment without information as to treatment). The safety population was comprised of the remaining 451 subjects who completed the prestudy period and took at least 1 dose of double-blind study drug. The intent-to-treat [ITT] efficacy population included 447 subjects, and the per-protocol [PP] efficacy population included 392 subjects (defined below). Completers for exposure (353 subjects) were defined as subjects who had at least 53 days of exposure to study drug.

On study day −1 (baseline), eligible subjects were randomly assigned to 1 of 2 fixed doses of ODV (50 or 100 mg/day) or to placebo for the 8-week double-blind treatment period. A 7-day taper period after the end of double-blind treatment was recommended but may have been omitted, shortened, or lengthened at the discretion of the investigator. Subjects assigned to the ODV 50 mg dose group received their maintenance dose beginning on study day 1 and continued this regimen until study day 56 or early withdrawal. For the taper period (days 1 through 7 after the end of the double-blind treatment period), they received 0 mg. Subjects assigned to the ODV 100 mg dose group were titrated to their maintenance dose. On study days 1 through 7 they received ODV 50 mg/day. Beginning on study day 8, they received their assigned dose of ODV 100 mg/day and continued on this regimen until study day 56 or early withdrawal. For the taper period (days 1 through 7 after the end of the double-blind treatment period), they received ODV 50 mg/day. Subjects assigned to placebo received their maintenance dose (0 mg) from study day 1 through day 56 or early withdrawal, and also received 0 mg during the taper period.

### Efficacy Evaluation

The primary outcome variable was the HAM-D₁₇ total score. The key secondary outcome variable was the Clinical Global Impressions Scale-Improvement (CGI-D score. Other secondary efficacy variables included the CGI-S, the Montgomery and Asberg Depression Rating Scale (MADRS) score, the HAM-D₆ (Bech version: HAM-D₁₇ items 1, 2, 7, 8, 10, and 13) score, the Covi Anxiety Scale total score, the remission rate (percentage of subjects with HAM-D₁₇ scores of ≦7), response rates for the HAM-D₁₇ (50% or greater change from baseline in the HAM-D₁₇ total score), MADRS, and CGI-I, and the visual analog scale for pain intensity (VAS-PI). Self-administered health outcomes assessments were measured by the Sheehan Disability Scale (SDS) and the World Health Organization 5-Item Well-Being Index (WHO-5).

Statistical analyses were based on the data from all individual clinical study sites. Unless otherwise stated, the use of the word “significant” in conjunction with the results refers to p-values≦0.05. All tests were 2-tailed. Because of the large number of sites with few subjects, data from individual sites were pooled (before the study was unblinded) to form groups with greater numbers of subjects. Each pooled group is referred to as a site for the purposes of the analysis. The ITT population, the primary population for efficacy analysis, included all subjects who were randomly assigned to treatment, had a baseline primary efficacy evaluation, took at least 1 dose of double-blind study drug, and had at least 1 primary efficacy evaluation after the first dose of double-blind study drug. The PP population included all subjects who were randomly assigned to treatment, had a baseline primary efficacy evaluation, took at least 1 dose of double-blind study drug, had at least 1 primary efficacy evaluation after the first dose of double-blind study drug, and had no major protocol violations. The all-randomized population included all subjects randomly assigned to treatment who had at least 1 baseline efficacy evaluation. The primary efficacy variable was the change from baseline in the HAM-D₁₇ total score, which was analyzed using analysis of covariance (ANCOVA) at the FOT evaluation (last-observation-carried-forward [LOCF] technique). Closed testing procedures were performed to compare the 2 doses (50 and 100 mg/day) of ODV with placebo based on the primary efficacy variable, the change in HAM-D₁₇ total score from baseline. A general linear model with multiple contrast statements was used to calculate F-statistics for the global null hypotheses and all intersection hypotheses. The closure principle was used to determine which hypothesis should have been retained or rejected at α=0.05. If a significant difference was detected for 1 or both doses of ODV, then a sequential testing method was applied to that dose(s) as follows: for 1 or both ODV dose group(s), if a significant difference from placebo on the primary efficacy variable was noted based on the closed testing procedure, the key secondary efficacy variable was tested at the 0.05 level to compare the ODV dose(s) with placebo. The HAM-D₁₇ change from baseline was also analyzed using a mixed-effects model with treatment, time, and the interaction of treatment and time as fixed effects, baseline HAM-D₁₇ total score as covariate, and site as a random effect. An autoregression of the first order (AR (1)) covariance matrix was used to model the within-subject errors. The ETRANK method, which corrects for missing data patterns, was also used to analyze changes in the HAM-D₁₇ scores from baseline (the primary efficacy variable). The CGI-1 score was the key secondary efficacy variable. Sequential testing was applied to the CGI-I. The order of testing was to first test the HAM-D₁₇. If a ODV treatment group was significantly different from placebo for the HAM-D₁₇, then the CGI-I was tested. The CGI-I score was analyzed as a categorical variable via the Cochran-Mantel-Haenszel test with treatment as the factor, controlling for site. The ridit scoring scheme, which yields a nonparametric analysis, was used. Mean scores on the CGI-I were also analyzed by ANOVA with treatment and site as factors. Other secondary variables include HAM-D₆ total score, MADRS total score, Covi Anxiety Scale total score, CGI-S, and the overall pain score and each subcomponent of VAS-PI. These variables were evaluated using ANCOVA on changes from baseline with treatment and site as factors and baseline value as the covariate. Remission, defined as a HAM-1)₁₇ total score of 7 or less, was analyzed using a logistic regression model with treatment and site as factors and the baseline HAM-D₁₇ total score value as the covariate. Response, defined as a decrease of 50% or more on the HAM-D₁₇ total score from baseline, was analyzed with the logistic regression model, with treatment and site as factors and baseline HAM-D₁₇ score as a covariate. Subjects who had CGI-I scores of 1 or 2 were classified as responders. These data were analyzed with the logistic regression model, with treatment and site as factors. Response on MADRS, defined as a decrease of 50% or more on the total score from baseline, was analyzed with the logistic regression model with treatment and site as factors and baseline MADRS score as a covariate. Efficacy analyses for the secondary variables were conducted at each time point using the LOCF technique and observed-cases data. No adjustment for multiplicity was made for the secondary efficacy variables.

The results of the administration for 50 mg and 100 mg doses are tabulated in Table 2. P-values of less than 0.05 are shown in bold.

TABLE 2

| | p-Value versus Placebo | |
|---|---|---|
| Efficacy Variable | 50 mg | 100 mg |
| Primary variable | | |
| HAM-D₁₇ total score | 0.018 | 0.065 |
| HAM-D₁₇ mixed model | <0.001 | 0.006 |
| Key secondary variable | | |
| CGI-I score (CMH) | 0.079 | 0.057 |
| CGI-I score (ANOVA) | 0.085 | 0.076 |
| Other secondary variables | | |
| MADRS total score (overall p = 0.060) | 0.022 | 0.095 |
| CGI-S score | 0.074 | 0.208 |
| HAM-D₁₇ response rate^{a} | 0.098 | 0.195 |
| HAM-D₁₇ remission rate^{b} | 0.027 | 0.090 |
| VAS-PI overall score (overall p = 0.119) | 0.233 | 0.041 |
| Stomach pain score | 0.736 | 0.233 |
| Back pain score (overall p = 0.065) | 0.785 | 0.031 |
| Chest pain score | 0.635 | 0.198 |
| Arms, legs, or joint pain score | 0.289 | 0.006 |

^{a}Response = 50% or greater reduction from baseline in HAM-D₁₇ total score.
^{b}Remission = HAM-D₁₇ total score of 7 or less.

The results of these analyses demonstrated the efficacy of the ODV 50 mg/day dose, and supported the efficacy of the ODV 100 mg/day dose, for the treatment of MDD. The antidepressant efficacy of the ODV 50 mg/day dose was superior to that of placebo based on ANCOVA (using LOCF technique) results for the primary efficacy variable (HAM-D₁₇ total score), and for 2 of the secondary variables (MADRS total and HAM-D₁₇ remission rate). For the HAM-D₁₇ total score at the FOT evaluation, the adjusted mean change from baseline was significantly (p=0.018) greater for the ODV 50 mg group (−11.5) in comparison with the placebo group (−9.53). Using the mixed-effects model analysis, the antidepressant efficacy of the ODV 100 mg/day dose was also superior to that of placebo for the HAM-D₁₇ total score. At the week 8 evaluation, the adjusted mean change from baseline was significantly greater for the ODV 50 mg group (−12.40; p<0.001) and the ODV 100 mg group (−11.88; p=006) in comparison with the placebo group (−9.86).

### Safety Evaluation

The safety of the two doses was determined using the following assessments: monitoring of adverse events (AEs), withdrawal because of AEs, concomitant medications, physical examinations, standard 12-lead electrocardiograms (ECGs), vital signs (supine and standing pulse rate and blood pressure (BP); body weight), laboratory determinations (hematology, blood chemistry, and urinalysis; free thyroxine index, including total thyroxine and triiodothyronine, and the β-HCG), and the Discontinuation-Emergent Signs and Symptoms (DESS) checklist.

The overall safety findings, for summary data as well as individual subject data, arc consistent with the safety results seen in other ODV studies. The most common (incidence≧5%) taper/post study-emergent AEs in the 50 mg group were diarrhea, nausea, abnormal dreams, dizziness, and hostility. The DESS checklist was administered to 340 subjects. Results of the analysis showed a significant difference (p=0.001) in the mean DESS scores in the 50 mg group compared with the placebo group during the first week of the taper period.

During the double-blind on-therapy period, 20% of the subjects in the safety population discontinued. AEs were the reason for study discontinuation in 3% of 152 subjects in the placebo group, 3% of 151 subjects in the 50 mg group, and 7% of 148 subjects in the 100 mg group. Serious or noteworthy AEs were reported by 9 (1.99%) of the 451 subjects in the safety population: 5 in the 50 mg group and 4 in the 100 mg group. No subject in the placebo group had a serious or noteworthy AE. AEs of clinical importance occurred in 27 (5.98%) of the subjects in the safety population: 4 in the placebo group, 13 in the 50 mg group (including subject who also had serious or noteworthy AE), and 10 in the 100 mg group.

Few subjects had blood chemistry, hematology, or lipid values that met the criteria for potential clinical importance. Of these, five ODV treated subjects (2 in the 50 mg group and 3 in the 100 mg group) had laboratory values that were considered clinically important by the medical monitor. Clinically important vital signs results were noted in four ODV treated subjects (3 with elevations in supine systolic or diastolic BP, and 1 with postural hypotension). Five other ODV treated subjects were identified with clinically important AEs related to vital signs results: hypertension in three subjects (2 in the 50 mg group and 1 in the 100 mg group), and hypotension in 2 subjects (both in the 50 mg group). None of the subjects had clinically important findings for ECGs or AEs of clinical importance related to ECG results.

### Example 3

### MDD Study No. 2

### Study Design

This example describes the results of a second multicenter, randomized, double-blind, placebo-controlled, parallel-group study that was also designed to evaluate the efficacy and safety of 50 and 100 mg/day ODV for treating MDD.

Subjects were male or female adults that met the same criteria as the subjects in Example 1. The planned enrollment was 450 subjects. A total of 565 subjects were screened for participation; 80 were screen failures and 485 were randomly assigned to treatment: 161 were assigned to receive placebo, 166 were assigned to receive ODV 50 mg/day; and 158 were assigned to receive ODV 100 mg/day. All 485 randomized subjects were included in the safety population (completed the prestudy period and took at least 1 dose of double-blind study drug). The intent-to-treat [ITT] efficacy population included 483 subjects, and the per-protocol [PP] efficacy population included 440 subjects. Completers for exposure (353 subjects) were defined as subjects who had at least 53 days of exposure to study drug.

The administration protocols for 50 or 100 mg/day and placebo groups were the same as in Example 1.

### Efficacy Evaluation

The primary and secondary outcomes were the same as in Example 1 as were the statistical methods. The results of the administration for 50 mg and 100 mg doses are tabulated in Table 3. P-values of less than 0.05 are shown in bold.

TABLE 3

| | p-Value versus Placebo | |
|---|---|---|
| Efficacy Variable | 50 mg | 100 mg |
| Primary variable | | |
| HAM-D₁₇ total score | 0.002 | <0.001 |
| HAM-D₁₇ mixed model | <0.001 | <0.001 |
| Key secondary variable | | |
| CGI-I score (CMH) | 0.002 | <0.001 |
| CGI-I score (ANOVA) | 0.003 | <0.001 |
| Other secondary variables | | |
| MADRS total score (overall p = 0.060) | 0.004 | <0.001 |
| CGI-S score | 0.003 | <0.001 |
| HAM-D₁₇ response rate^{a} | 0.004 | 0.011 |
| HAM-D₁₇ remission rate^{b} | 0.099 | 0.002 |

^{a}Response = 50% or greater reduction from baseline in HAM-D₁₇ total score.
^{b}Remission = HAM-D₁₇ total score of 7 or less.

The antidepressant efficacy of ODV 50 and 100 mg/day was superior to that of placebo based on ANCOVA (using LOCF technique) results for the primary efficacy variable (HAM-D₁₇ total score), the key secondary variable (CGI-I score), and the other secondary variables (except HAM-D₁₇ remission rate for the DVS SR 50 mg/day group). For the HAM-D₁₇ total score at the FOT evaluation, the adjusted mean change from baseline was significantly greater for the 50 mg group (−13.2; p=0.002) and the 100 mg group (−13.7; p<0.001) compared with the placebo group (−10.7). Using the mixed effect model analysis, both doses were superior to placebo for the HAM-D₁₇ total score. At the week 8 evaluation, the adjusted mean change from baseline was significantly greater (p<0.001) for the 50 mg group (−14.4) and the 100 mg group (−14.9) compared with the placebo group (−11.5).

### Safety Evaluation

The safety of the two doses was determined using the same assessments as in Example 1. The most common (incidence≧5%) taper/poststudy-emergent AEs in the 50 mg group were headache, nausea, dizziness, and insomnia. The most common (incidence≧5%) TPAEs in the 100 mg group were headache, nausea, depression, dizziness, and vertigo. Headache was the only TPAE with an incidence≧5% in the placebo group.

The DESS checklist was used to evaluate symptoms that first occurred, or that worsened, during the taper period (the 7-day period after the end of the double-blind treatment period). During this 7-day period, doses of ODV were tapered to 0 mg for subjects in the 50 mg group, and to 50 mg for subjects in the 100 mg group. The DESS checklist was administered to 420 of the 423 subjects who had completed at least 53 days of on-therapy treatment. Results of the analysis for the 50 mg group showed a significant (p=0.001) difference in mean DESS scores in comparison with the placebo group during the 7-day taper period. Results of the analysis for the 100 mg group showed a significant (p=0.017) difference in the mean DESS in comparison with the placebo group during the week after the 7-day taper period.

During the on-therapy period, 5% of the 485 subjects in the safety population discontinued from the study. AEs were the reason for discontinuation in 3% of subjects in the placebo group, 5% of subjects in the 50 mg group, and 7% of subjects in the 100 mg group. AEs that led to discontinuation at an incidence >1% in the 50 mg group were nausea (1.2%), sweating (1.2%), and vomiting (1.2%). In the 100 mg group the AEs that led to discontinuation at an incidence >1% were asthenia (1.3%), headache (1.3%), nausea (3.8%), and anorgasmia in men (2.1%). A serious adverse event (SAE) occurred in 1 subject during the prestudy period before the subject had been randomly assigned to treatment. SAEs were also reported during the poststudy period by 2 of the 485 subjects (<1%) in the safety population. None of the subjects had SAEs during the on-therapy period. None of the subjects had noteworthy AEs. No deaths occurred during this study and none were subsequently reported. AEs of clinical importance were reported by 29 of the 485 subjects (5%) in the safety population during the on-therapy or poststudy periods. The majority of these events were either changes in BP or benign episodes of irritability-like symptoms that occurred during the taper or poststudy period and that were categorized as hostility by COSTART. Few subjects had blood chemistry, hematology, or lipid values that met the criteria for potential clinical importance. Liver function test results considered of clinical importance by the medical monitor occurred in less than 1% of the 485 subjects in the safety population (1 subject in the placebo group and 1 subject in the 50 mg group had elevated SGOT/AST levels, and another subject in the 50 mg group had elevated SGPT/ALT levels). There were no cases of liver failure. Elevated lipid/triglyceride values considered of clinical importance by the medical monitor also occurred in less than 1% of the 485 subjects in the safety population (1 subject in the 50 mg group had increased total cholesterol levels). Although 8.8% of the 432 subjects tested for urine protein had values of potential clinical importance, the medical monitor did not identify any subject with proteinuria of clinical importance.

### Example 4

### MDD Study No. 3

### Study Design

This example describes the results of a third multicenter, randomized, double-blind, placebo-controlled, parallel-group study that was also designed to evaluate the efficacy and safety of 50 and 100 mg/day ODV for treating MDD.

Subjects were male or female adults that met the same criteria as the subjects in Example 1. The administration protocols for 50 or 100 mg/day and placebo groups were the same as in Example 1.

### Efficacy Evaluation

The primary and secondary outcomes were the same as in Example 1 as were the statistical methods. A Global F-test was also used to analyze the results. The purpose of this test was to compare the two fixed doses with placebo to control for Type-I error. Statistical significance is met if the p-value for the Global F-test <0.05, followed by un-adjusted pairwise comparison between either dose and placebo at 0.05 significance level. This study did not meet statistical significance using this test (p=0.086).

The results of the administration for 50 mg and 100 mg doses are tabulated in Tables 4 and 5. P-values of less than 0.05 are shown in bold. When unadjusted (nominal) p-values were examined, the 100 mg group but not the 50 mg group separated from placebo using the primary efficacy variable (HAM-D₁₇ total score) and the key secondary variable (CGI-I score).

TABLE 4

| | Diff. versus | | p-Value | |
|---|---|---|---|---|
| | Placebo (95% CI) | | versus Placebo | |
| Efficacy Variable | 50 mg | 100 mg | 50 mg | 100 mg |
| HAM-D₁₇ total score | | | | |
| LOCF^{a} | 1.1 (−0.6, 2.7) | 1.8 (0.2, 3.4) | 0.198 | 0.028 |
| MMRM^{b} | 1.4 (−0.0, 2.8) | 2.2 (0.7, 3.6) | 0.056 | 0.003 |
| OC^{c} | 1.8 (−0.1, 3.6) | 2.9 (1.0, 4.7) | 0.062 | 0.003 |

TABLE 5

| | | Baseline | Δ from | p-Value versus |
|---|---|---|---|---|
| Efficacy Variable | Treatment | Score | Baseline | Placebo |
| CGI-I score (CMH) | Placebo | — | — | — |
| | 50 mg | — | — | 0.110 |
| | 100 mg | — | — | 0.009 |
| CGI-S score | Placebo | 4.0 | −1.10 | — |
| | 50 mg | 4.3 | −1.25 | 0.248 |
| | 100 mg | 4.3 | −1.44 | 0.011 |
| HAM-D₆ | Placebo | 13.0 | −4.82 | — |
| | 50 mg | 12.8 | −5.41 | 0.215 |
| | 100 mg | 12.9 | −6.15 | 0.005 |
| MADRS total score | Placebo | 31.1 | −11.0 | — |
| | 50 mg | 30.1 | −12.7 | 0.149 |
| | 100 mg | 30.0 | −14.4 | 0.004 |

### Safety Evaluation

The safety of the two doses was determined using the same assessments as in Example 1. The distribution and reasons for discontinuation in each group are set forth in Table 6.

TABLE 6

| | Placebo | 50 mg | 100 mg |
|---|---|---|---|
| Reason | n = 161 | n = 148 | n = 150 |
| Discontinued (total) | 38 (24) | 28 (19) | 33 (22) |
| Adverse Event | 10 (6) | 8 (5) | 11 (7) |
| Failed to Return | 1 (1) | 3 (2) | 2 (1) |
| Investigator Request | 1 (1) | 0 | 0 |
| Lost to Follow-up | 6 (4) | 12 (8) | 7 (5) |
| Other | 2 (1) | 0 | 0 |
| Protocol Violation | 5 (3) | 0 | 5 (3) |
| Subject Request | 6 (3) | 2 (1) | 5 (3) |
| Unsatisfactory Response-Efficacy | 7 (4) | 3 (2) | 3 (2) |

The most common (incidence >5%) taper/poststudy-emergent AEs are summarized in Table 7.

TABLE 7

| | Placebo | 50 mg | 100 mg |
|---|---|---|---|
| | n = 161 | n = 148 | n = 150 |
| | n(F) = 94 | n(F) = 102 | n(F) = 99 |
| Adverse Event | n(M) = 67 | n(M) = 46 | n(M) = 51 |
| Asthenia | 6 (4) | 12 (8) | 18 (12) |
| Flu Syndrome | 5 (3) | 6 (4) | 10 (7) |
| Anorexia | 5 (3) | 14 (10) | 14 (9) |
| Constipation | 4 (3) | 9 (6) | 10 (7) |
| Nausea | 14 (9) | 33 (22) | 35 (23) |
| Vomiting | 3 (2) | 2 (1) | 6 (4) |
| Abnormal Dreams | 3 (2) | 5 (3) | 4 (3) |
| Insomnia | 6 (4) | 20 (14) | 26 (17) |
| Somnolence | 8 (5) | 14 (10) | 18 (12) |

### OTHER EMBODIMENTS

The foregoing has been a description of certain non-limiting preferred embodiments of the invention. Those of ordinary skill in the art will appreciate that various changes and modifications to this description may be made without departing from the spirit or scope of the present invention, as defined in the following claims.

We claim:

1. A method of treating a patient suffering from major depressive disorder, the method comprising administering to a patient in need thereof a daily dose of about 50 mg O-desmethyl-venlafaxine or an equivalent amount of a pharmaceutically acceptable salt thereof.

2. The method of claim 1, wherein the daily dose is administered as an oral dosage form comprising O-desmethyl-venlafaxine or an equivalent amount of a pharmaceutically acceptable salt thereof and one or more pharmaceutically acceptable excipients.

3. The method of claim 2, wherein the oral dosage form comprises O-desmethyl-venlafaxinc succinate.

4. The method of claim 3, wherein the oral dosage form is a sustained release oral dosage form.

5. The method of claim 4, wherein the oral dosage form is a tablet.

6. The method of claim 4, wherein the oral dosage form comprises a rate controlling polymer.

7. The method of claim 6, wherein the rate controlling polymer is selected from hydroxyalkyl celluloses, poly(ethylene)oxides, alkyl celluloses, carboxymethyl celluloses, hydrophilic cellulose derivatives, and polyethylene glycol.

8. The method of claim 6, wherein the rate controlling polymer is hydroxypropyl methyl cellulose.

9. The method of claim 6, wherein the oral dosage form comprises from about 10% to about 30% by weight of O-desmethyl-venlafaxine succinate and from about 50% to about 70% by weight of the rate controlling polymer, based upon total weight of oral dosage form.

10. The method of claim 3, wherein the oral dosage form comprises a filler.

11. The method of claim 10, wherein the filler is microcrystalline cellulose.

12. The method of claim 10, wherein the oral dosage form comprises from about 10% to about 30% by weight of O-desmethyl-venlafaxine succinate and from about 5% to about 15% by weight of the filler, based upon total weight of oral dosage form.

13. The method of claim 3, wherein the oral dosage form comprises a lubricant.

14. The method of claim 13, wherein the lubricant is magnesium stearate.

15. The method of claim 13, wherein the oral dosage form comprises from about 10% to about 30% by weight of O-desmethyl-venlafaxine succinate and from about 0.5% to about 2% by weight of the lubricant, based upon total weight of oral dosage form.

16. The method of claim 3, wherein the oral dosage form comprises a glidant.

17. The method of claim 16, wherein the glidant is talc.

18. The method of claim 16, wherein the oral dosage form comprises from about 10% to about 30% by weight of O-desmethyl-venlafaxine succinate and from about 2% to about 4% by weight of the glidant, based upon total weight of oral dosage form.

19. The method of claim 3, wherein the oral dosage form comprises:
from about 10% to about 30% by weight of O-desmethyl-venlafaxine succinate;
from about 50% to about 70% by weight of the rate controlling polymer;
from about 5% to about 15% by weight of the filler;
from about 0.5% to about 2% by weight of the lubricant; and
from about 2% to about 4% by weight of the glidant, based upon total weight of oral dosage form.

20. The method of claim 19, wherein the rate controlling polymer is hydroxypropyl methyl cellulose, the filler is microcrystalline cellulose, the lubricant is magnesium stearate, and the glidant is talc.

21. The method of claim 1, wherein the patient is characterized by a primary diagnosis of MDD.

22. The method of claim 2, wherein the oral dosage form is administered as a single dose.

23. The method of claim 22, wherein the oral dosage form comprises 50 mg O-desmethyl-venlafaxine or an equivalent amount of a pharmaceutically acceptable salt thereof.

24. The method of claim 23, wherein the oral dosage form comprises O-desmethyl-venlafaxine succinate.

25. The method of claim 1, wherein the daily dose of about 50 mg is administered during a treatment period and the step of administering further comprises administering a gradually decreasing daily dose during a tapering period which follows the treatment period.

26. The method of claim 25, wherein the tapering period lasts for between 4 and 10 days.

27. The method of claim 1, wherein the daily dose of about 50 mg is administered during a treatment period and the step of administering further comprises administering a gradually increasing daily dose during a titration period which precedes the treatment period.

28. The method of claim 27, wherein the titration period lasts for between 4 and 10 days.

29. The method of claim 1, wherein the patient suffers from severe renal impairment and/or end-stage renal disease and the daily dose is administered every other day.

30. The method of claim 1, wherein the patient has previously been administered a monoamine oxidase inhibitor, the method further comprising waiting 14 days before administering a first daily dose of O-desmethyl-venlafaxine.

31. The method of claim 1, wherein the patient is subsequently administered a monoamine oxidase inhibitor, the method further comprising waiting 7 days before administering the first dose of monoamine oxidase inhibitor.
